(19) United States
(12) Patent Application Publication
(10) Pub. No.: US 2025/0196082 A1
(43) Pub. Date: Jun. 19, 2025

(54) MIXING BAG FOR CELL PRESERVATION SYSTEMS

(71) Applicant: Terumo BCT, Inc., Lakewood, CO (US)

(72) Inventor: Nathaniel T. JOHNSON, Highlands Ranch, CO (US)

(21) Appl. No.: 18/977,120

(22) Filed: Dec. 11, 2024

Related U.S. Application Data
(60) Provisional application No. 63/610,684, filed on Dec. 15, 2023.

(51) Int. Cl. B01F 35/513; B01F 31/29; B01F 35/40; B01F 101/44; C12N 5/00

(52) U.S. Cl. CPC B01F 35/513; B01F 31/29; B01F 35/40; C12N 5/545; B01F 2101/44

(57) ABSTRACT

A bag for mixing fluid volumes includes a port at a center of a first end, a second end, a first constriction, and a second constriction. The port is configured to couple to at least one fluid line for introducing and removing fluids from the bag. The second end includes a first lobe and a second lobe. The first constriction is on the first side between the first end and the second end and the second constriction is on a second side between the first end and the second end.

### CROSS-REFERENCE TO RELATED APPLICATIONS

This application claims the benefit of U.S. Provisional Application No. 63/610,684 filed on Dec. 15, 2023. The entire disclosure of which is incorporated herein by reference.

### FIELD

The present disclosure relates to mixing bags for cell preservation systems.

### BACKGROUND

This section provides background information related to the present disclosure which is not necessarily prior art.

Cell Expansion Systems (CESs) are used to expand and differentiate cells. Cell expansion systems may be used to expand, e.g., grow, a variety of adherent and suspension cells. After expansion, the cells must be preserved and transported for use. Cells may be mixed with one or more preservation agents to prepare the cells for transport.

### SUMMARY

This section provides a general summary of the disclosure and is not a comprehensive disclosure of its full scope or all of its features.

At least one example embodiment includes a bag for mixing fluid volumes. The bag may include a port at a center of a first end, a second end, a first constriction, and a second constriction. The port may be configured to couple to at least one fluid line for introducing and removing fluids from the bag. The second end may include a first lobe and a second lobe. The first constriction may be on the first side between the first end and the second end and the second constriction may be on a second side between the first end and the second end.

In at least one example embodiment, the first end may be tapered to the port from the first side and the second side.

In at least one example embodiment, the first lobe and the second lobe may be circular.

In at least one example embodiment, the bag may further include a connection at a center of the second end between the first lobe and the second lobe. The connection may be configured to couple with a hanger of a collection system to hold the bag within the collection system. In at least one example embodiment, the first lobe may be configured to extend above the connection towards the first side and the second lobe may be configured to extend above the connection towards the second side.

In at least one example embodiment, the first constriction may include a first portion and a second portion. Each of the first portion and the second portion may be configured to taper from the second end to the first end of the bag.

In at least one example embodiment, the second constriction may include a first portion and a second portion. Each of the first portion and the second portion may be configured to taper from the second end to the first end of the bag.

Also described herein is a system for mixing fluid volumes. The system may include a bag, a hanger, and at least one mixing paddle. The bag may include a port at a center of a first end, a second end, a first constriction, and a second constriction. The port may be configured to couple to at least one fluid line for introducing and removing fluids from the bag. The second end may include a first lobe and a second lobe. The first constriction may be on the first side between the first end and the second end and the second constriction may be on a second side between the first end and the second end. The hanger may be configured to couple to the second end of the bag to hold the bag in the system. The at least one mixing paddle may be configured to interact with the bag to mix the fluid volumes within the bag.

In at least one example embodiment, the first end may be tapered to the port from the first side and the second side.

In at least one example embodiment, the first lobe and the second lobe may be circular.

In at least one example embodiment, the bag may further include a connection at a center of the second end between the first lobe and the second lobe. The connection may be configured to couple with a hanger of a collection system to hold the bag within the collection system. In at least one example embodiment, the first lobe may be configured to extend above the connection towards the first side and the second lobe may be configured to extend above the connection towards the second side.

In at least one example embodiment, the first constriction may include a first portion and a second portion. Each of the first portion and the second portion may be configured to taper from the second end to the first end of the bag.

In at least one example embodiment, the second constriction may include a first portion and a second portion. Each of the first portion and the second portion may be configured to taper from the second end to the first end of the bag.

In at least one example embodiment, the at least one mixing paddle may include a first mixing paddle and a second mixing paddle. In at least one example embodiment, the first mixing paddle may be configured to pinch the bag to a plate of the system at a central location. In at least one example embodiment, the central location may be between the first constriction and the second constriction such that a first flow path is created between the first constriction and the first mixing paddle and a second flow path is created between the second constriction and the first mixing paddle. In at least one example embodiment, the second mixing paddle may be located between the first mixing paddle and the first end of the bag when the bag is attached to the hanger. In at least one example embodiment, the second mixing paddle may be configured to move from a first location to a second location relative to the bag to mix the fluid volumes within the bag.

Also described herein is a cell preservation system. The cell preservation system may include a cooling plate, at least one mixing paddle, and at least one tab. The cooling plate may be configured to be cooled to a mixing temperature during a mixing process. The at least one mixing paddle may be configured to mix fluids within a mixing bag during the mixing process. The at least one tab may be configured to overlap with a first side of the cooling plate. The at least one tab may be configured to be disposed between at least a portion of the mixing bag and the cooling pate during the mixing process.

In at least one example embodiment, the at least one tab may include a first tab and a second tab. In at least one example embodiment, the first tab may be disposed on the first side of the cooling plate and the second tab may be disposed on a second side of the cooling plate.

In at least one example embodiment, the at least one tab may include a connection point proximate to the cooling plate. In at least one example embodiment, the at least one tab ay extend from the connection point onto the first side of the cooling plate. In at least one example embodiment, the at least one tab may extend from the connection point onto the first side of the cooling plate at a first angle.

In at least one example embodiment, the cell preservation system may further include a hanger configured to hold the mixing bag in place during the mixing process. In at least one example embodiment, the at least one tab may be disposed between the hanger and the at least one mixing paddle.

In at least one example embodiment, the at least one tab may be configured to extend from a connection point proximate to the cooling plate towards the at least one mixing paddle.

In at least one example embodiment, the at least one mixing paddle may include at least one cut-out configured to extend a first distance from the cooling plate when the at least one mixing paddle is in a compressed state. In at least one example embodiment, the at least one tab may be configured to extend at least partially through the at least one cut-out of the at least one mixing paddle.

In at least one example embodiment, the at least one tab may be configured to prevent the mixing bag from touching the cooling plate at a location of the at least one tab. In at least one example embodiment, the location may be a portion of the mixing bag that does not contain fluid.

In at least one example embodiment, the at least one tab may have a length of about 60 mm.

In at least one example embodiment, the at least one tab may have a width of about 5 mm.

In at least one example embodiment, the at least one tab may include plastic.

### DRAWINGS

The drawings described herein are for illustrative purposes only of selected embodiments and not all possible implementations and are not intended to limit the scope of the present disclosure.

FIG. 1 illustrates a cell preservation system according to at least one example embodiment.

FIG. 2 illustrates a portion of the cell preservation system of FIG. 1 including a cooling plate and at least one tab according to at least one example embodiment.

FIG. 3 illustrates the cooling plate and a tab of FIG. 2 with a mixing paddle in a compressed state according to at least one example embodiment.

FIG. 4 illustrates the at least one tab of FIG. 2 according to at least one example embodiment.

FIG. 5 illustrates a portion of the cell preservation system of FIG. 1 coupled to a mixing bag containing fluids according to at least one example embodiment.

FIG. 6 illustrates the mixing bag of FIG. 5 according to at least one example embodiment.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, in component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Various components are referred to herein as “operably associated.” As used herein, “operably associated” refers to components that are linked together in operable fashion and encompasses embodiments in which components are linked directly, as well as embodiments in which additional components are placed between the linked components. “Operably associated” components can be “fluidly associated.” “Fluidly associated” refers to components that are linked together such that fluid can be transported between them. “Fluidly associated” encompasses embodiments in which additional components are disposed between the two fluidly associated components, as well as components that are directly connected. Fluidly associated components can include components that do not contact fluid, but contact other components to manipulate the system (e.g., a peristaltic pump that pumps fluids through flexible tubing by compressing the exterior of the tube).

The term “donor,” as used herein, can mean any person providing a fluid (e.g., whole blood) to the apheresis system. A donor can also be a patient that also provides a fluid to the apheresis system temporarily while the fluid is processed, treated, manipulated, etc. before being provided back to the patient.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material”.

The term “computer-readable medium” as used herein refers to any tangible storage and/or transmission medium that participates in providing instructions to a processor for execution. Such a medium may take many forms, including but not limited to, non-volatile media, volatile media, and transmission media. Non-volatile media includes, for example, NVRAM, or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a CD-ROM, any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored.

The term “module” as used herein refers to any known or later developed hardware, software, firmware, artificial intelligence, fuzzy logic, or combination of hardware and software that is capable of performing the functionality associated with that element.

The terms “determine”, “calculate” and “compute,” and variations thereof, as used herein, are used interchangeably and include any type of methodology, process, mathematical operation or technique.

Example embodiments of the present disclosure are generally directed to elements of cell preservation systems and mixing bags for cell preservation systems. Cell preservation systems are configured to receive cells, potentially cells from a cell expansion system, and prepare the cells for transportation for later use. The cells must be preserved in order to prepare the cells for transport. In at least one example embodiment, the cells are preserved using cryopreservation techniques.

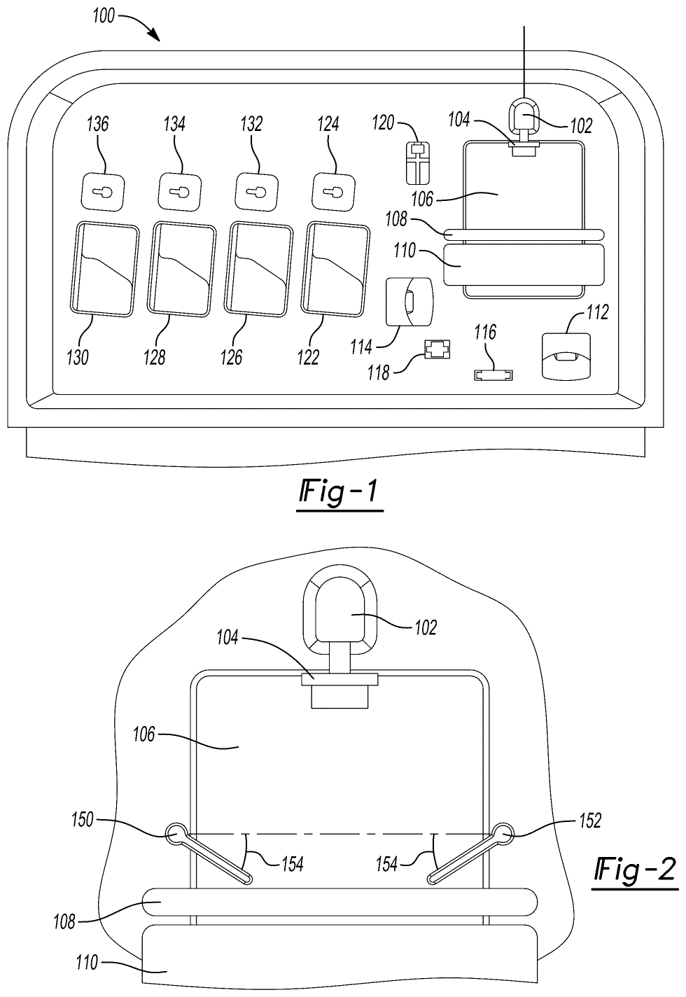

FIG. 1 is an example embodiment of a cell preservation system 100. One or more disposables may interact with the cell preservation system 100 as described in further detail below. The cell preservation system 100 includes a load sensor 102 that includes a hanger 104. In at least one example embodiment, the hanger 104 may be a hook such as a linear hook. A mixing bag may be held within the cell preservation system 100 by the hanger 104 and the load sensor 102 may be configured to weigh the mixing bag while it is held by the hanger 104. The cell preservation system 100 may also include a cooling plate 106. The cooling plate 106 may also be referred to herein as a mixing bag cooling plate. The cooling plate 106 may be cooled to a predetermined temperature so ensure that the cell mixture of the mixing bag is cooled to a specified temperature. The cell preservation system 100 may also include a first mixing paddle 108 and a second mixing paddle 110. The first mixing paddle 108 may be positioned closer to the load sensor 102 than the second mixing paddle 110. The first mixing paddle 108 may be configured to hold the mixing bag in place when the cell preservation system 100 is in use. For example, the first mixing paddle 108 may be configured to compress such that it is pressing the mixing bag together against the cooling plate 106 to prevent movement of the mixing bag during use of the cell preservation system 100. The second mixing paddle 110 may be configured to compress and extend relative to the cooling plate 106 to mix the cell mixture within the mixing bag during use of the cell preservation system 100. Thus, when in use, a mixing bag is placed between the cooling plate 106 and the first mixing paddle 108 and the second mixing paddle 110. The mixing bag is held in place both by the hanger 104 and the first mixing paddle 108 during operation of the cell preservation system 100.

The cell preservation system 100 may also include a supply pump 112 and a product pump 114. The supply pump 112 may be configured to pump the cell mixture into the mixing bag. In at least one example embodiment, the cell mixture may include cells such as cells grown from a cell expansion system and one or more preservation agents to be mixed with the cells. The product pump 114 may be configured to pump the cell mixture from the mixing bag into a quality control bag and one or more product bags after completion of mixing by the cell preservation system.

The cell preservation system 100 may also include a first tubing holder 116 and a second tubing holder 118. The first tubing holder 116 and the second tubing holder 118 may be configured to hold tubing in place when the cell preservation system 100 is in use. The first tubing holder 116 and the second tubing holder 118 may also prevent tubing lines from being twisted or kinked during use of the cell preservation system 100.

The cell preservation system 100 may also include at least one fluid sensor 120. The at least one fluid sensor 120 may be configured to detect fluid within a fluid line leading from the mixing bag to the quality control bag or the one or more product bags.

As described above, the disposables configured to interact with the cell preservation system may include a quality control bag and one or more product bags. The quality control bag may be disposed in a quality control bag holder 122 and may interact with a quality control valve 124 that may be configured to direct the cell mixture from the mixing bag to the quality control bag. The quality control valve 124 may also be configured to seal the quality control bag after the quality control bag is full of the cell mixture from the mixing bag.

The cell preservation system 100 may also include a first product bag holder 126, a second product bag holder 128, and a third product bag holder 130. A first product bag may be disposed in the first product bag holder 126, a second product bag may be disposed in the second product bag holder 128, and a third product bag may be disposed in the third product bag holder 130 during operation of the cell preservation system 100. The cell preservation system 100 may also include a first product valve 132, a second product valve 134, and a third product valve 136. Similar to the quality control valve 124, the first product valve 132, the second product valve 134, and the third product valve 136 may be configured to direct the cell mixture from the mixing bag to the corresponding first product bag, second product bag, or third product bag. The first product valve 132, the second product valve 134, and the third product valve 136 may also be configured to seal the corresponding first product bag, second product bag, or third product bag after the product bags are full of the cell mixture from the mixing bag. In at least one example embodiment, the quality control bag holder 122, the first product bag holder 126, the second product bag holder 128, and the third product bag holder 130 may be configured to maintain the cell mixture disposed in the corresponding quality control bag, first product bag, second product bag, or third product bag at the desired temperature while preparing the cell mixture for transportation to a final destination.

FIG. 2 illustrates the cooling plate 106, the first mixing paddle 108, the second mixing paddle 110, a first tab 150, and a second tab 152. In at least one example embodiment, the cooling plate 106 may accumulate condensation as the cooling plate 106 is cooled to a desired temperature. In at least one example embodiment, the desired temperature may be between about 4° C. and about 12° C., although example embodiments are not limited herein. As condensation accumulates on the cooling plate 106, a mixing bag held by the hanger 104 may stick to the cooling plate 106. If the mixing bag is stuck or partially stuck to the cooling plate 106 via the condensation, the mixing bag may be incorrectly weighed which may impact function of the cell preservation system 100. In at least one example embodiment, the first tab 150 and the second tab 152 may be configured to prevent the mixing bag from touching the cooling plate 106 at a location of the mixing bag that does not include fluid.

The first tab 150 and the second tab 152 may be stationary with respect to the cooling plate 106. An angle 154 between the first tab 150 and the second tab 152 and horizontal may be approximately 40°. However, the angle 154 is not limited herein and may be adjusted based on dimensions of the mixing bag to maximize overlap with the mixing bag in at least one example embodiment.

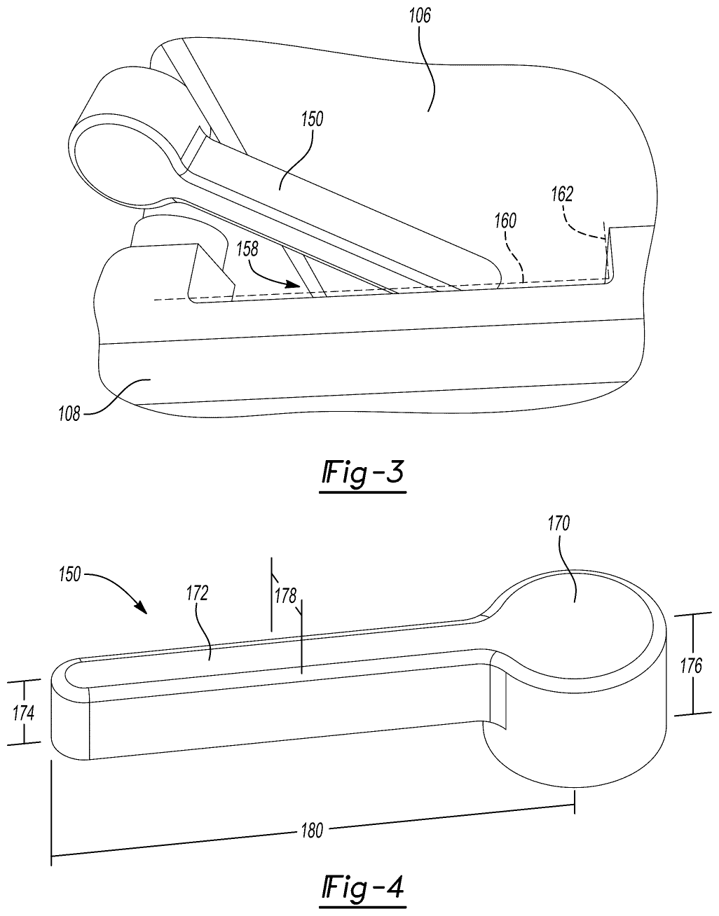

FIG. 3 illustrates the first tab 150 located between the first mixing paddle 108 and the cooling plate 106 when the first mixing paddle 108 is compressed against the cooling plate 106. In at least one example embodiment, the first mixing paddle 108 may have an area 158 with a width 160 of approximately 60 mm and a depth of approximately 9 mm that is not touching the cooling plate 106. The area 158 may have a height of approximately 20 mm. The first tab 150 may extend through the area 158 such that the first tab 150 is between the cooling plate 106 and a mixing bag when the mixing bag is coupled to the cell preservation system 100. The area 158 may be symmetric about a center of the cooling plate 106 and the second tab 152 may extend through a similar area between the cooling plate 106 and the first mixing paddle 108. The first tab 150 and the second tab 152 may be configured to provide separation between the mixing bag and the cooling plate 106 at a portion of the mixing bag where fluid flows when the first mixing paddle 108 is compressed.

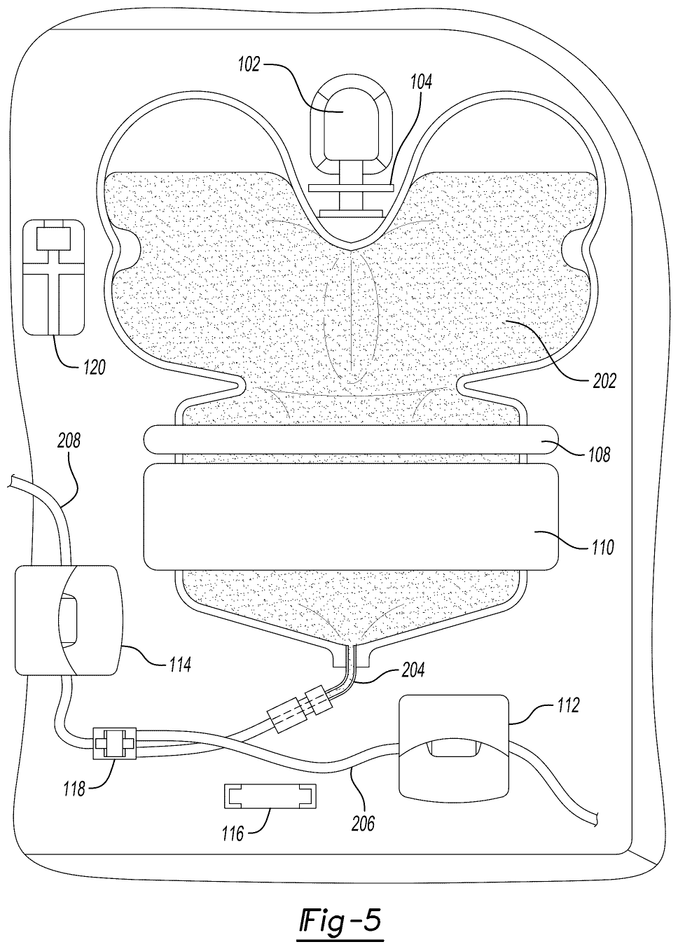

FIG. 4 illustrates an example embodiment of the first tab 150 and the second tab 152. FIG. 4 will be described with reference to the first tab 150 but the dimensions are uniform between the first tab 150 and the second tab 152. The dimensions for the first tab 150 described herein are not limiting and may be adjusted based on the cell preservation system 100 and the mixing bag. In at least one example embodiment, the first tab 150 may have a boss 170. The boss 170 may couple to the cell preservation system 100 adjacent to the cooling plate 106 and a protruding portion 172 of the first tab 150 may extend onto the cooling plate 106.

The protruding portion 172 of first tab 150 may have a height 174 of approximately 6.5 mm and the boss 170 may have a height 176 of approximately 10 mm. The protruding portion 172 of first tab 150 may have a width 178 of approximately 5 mm. A length 180 from a center of the boss 170 to an end of the protruding portion 172 opposite the boss 170 may be approximately 60 mm.

In at least one example embodiment, the first tab 150 and the second tab 152 may be formed from plastic such as polycarbonate, acrylonitrile butadiene styrene, nylon, polyethylene terephthalate, high density polyethylene, polyvinyl chloride, or polypropylene. However, the materials described herein for the first tab 150 and the second tab 152 are not limiting.

FIG. 5 is an illustration of a portion of the cell preservation system 100 in operation with a mixing bag 202. The mixing bag 202 is full of a cell mixture that is mixed by the first mixing paddle 108 and the second mixing paddle 110. The mixing bag 202 may include a mixing bag fluid line 204 that may be coupled to the second tubing holder 118. The second tubing holder 118 may be a Y-connector holder that may connect the mixing bag fluid line 204 to a supply fluid line 206 and a distribution fluid line 208. The supply fluid line 206 may be configured to supply fluid into the mixing bag 202 and the distribution fluid line 208 may be configured to supply fluid from the mixing bag 202 to the quality control bag and the one or more product bags as described above.

The mixing bag 202 may be held in place against the cooling plate 106 by the first mixing paddle 108 and the second mixing paddle 110 may be configured to compress and extend relative to the cooling plate 106 to mix the fluid within the mixing bag 202. The first mixing paddle 108 may compress a central area of the mixing bag 202 and fluid within the mixing bag 202 may flow along a first flow path and a second flow path located on opposites sides of the first mixing paddle 108 to provide fluid flow throughout the mixing bag 202. The first flow path may be through a portion of the mixing bag 202 located in the area 158 and the second flow path may be through a portion of the mixing bag 202 located in an area on an opposite side of the mixing bag 202.

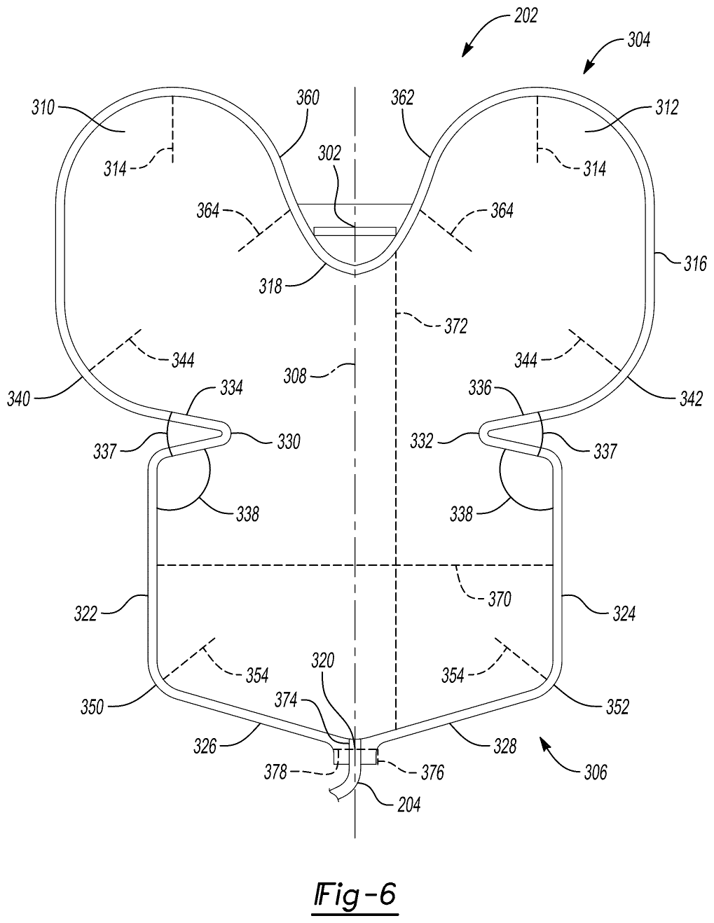

FIG. 6 is a front view of the mixing bag 202. The hanger 104 may hold the mixing bag 202 in place in the cell preservation system at a connection 302. In at least one example embodiment, the connection 302 may be an opening in the mixing bag 202 that the hanger 104 may be configured to interact with to hold the mixing bag 202 in place. Thus, a first end 304 of the mixing bag 202 may be proximate to the hanger 104 when the mixing bag 202 is loaded into the cell preservation system 100. The mixing bag 202 may also have a second end 306 opposite the first end 304. The second end 306 may be coupled to the mixing bag fluid line 204.

In at least one example embodiment, the mixing bag 202 may be symmetric about a vertical center line 308. Thus, each side of the mixing bag 202 is configured to facilitate uniform mixing of a cell mixture during operation of the cell preservation system 100.

The first end 304 of the mixing bag 202 may include a first lobe 310 and a second lobe 312. The first lobe 310 and the second lobe 312 are circular to allow fluid to sweep around the first lobe 310 and the second lobe 312 during operation of the cell preservation system 100. The first lobe 310 and the second lobe 312 may each extend above the connection 302. In at least one example embodiment, each of the first lobe 310 and the second lobe 312 may have a radius 314 of approximately 47 mm. However, example embodiments are not limited herein and the radius of the first lobe 310 and the second lobe 312 may be adjusted based on constraints of the cell preservation system 100 such as a location of the hanger 104 and the first mixing paddle 108 and the second mixing paddle 110.

A perimeter 316 of the mixing bag 202 may taper from the first lobe 310 and the second lobe 312 into a concave center portion 318 proximate to the vertical center line 308 if the mixing bag 202. Thus, when fluid is being mixed in the mixing bag 202, it may flow from the second end 306 of the bag towards the concave center portion 318 which may help to divide the fluid into the first lobe 310 and the second lobe 312 to create uniform mixing of the fluid. In at least one example embodiment, uniform mixing of the fluid may be mixing that eliminates stagnation points within the fluid.

The second end 306 of the mixing bag 202 may be tapered towards a connection point 320 where the mixing bag 202 couples to the mixing bag fluid line 204. In particular, the mixing bag 202 may have a first edge 322 on a first side of the mixing bag 202 and a second edge 324 on a second side of the mixing bag 202. The first edge 322 and the second edge 324 may be substantially vertical and may couple to a first tapered edge 326 and a second tapered edge 328, respectively. The first edge 322 and the first tapered edge 326 may be symmetric with the second edge 324 and the second tapered edge 328 about the vertical center line 308. The first tapered edge 326 and the second tapered edge 328 may funnel fluid from the mixing bag 202 to the mixing bag fluid line 204 when fluid is removed from the mixing bag 202. In at least one example embodiment, the first tapered edge 326 and the second tapered edge 328 may be angled about 110 degrees from the first edge 322 and the second edge 324, respectively.

The first edge 322 and the second edge 324 may abut a first triangular feature 330 and a second triangular feature 332, respectively. The first triangular feature 330 may also be referred to herein as a first constriction and the second triangular features 332 may be referred to herein as a second constriction. The first triangular feature 330 and the second triangular feature 332 act as constriction points of the mixing bag 202 and are above the first mixing paddle 108 and the second mixing paddle 110 when the mixing bag 202 is installed within the cell preservation system 100. The first triangular feature 330 and the second triangular feature 332 increase the velocity of the fluid as the fluid moves from the second end 306 of the mixing bag 202 to the first end 304 of the mixing bag 202. In particular, as the second mixing paddle 110 moves relative to the mixing bag 202, fluid within the mixing bag 202 is displaced. As the second mixing paddle 110 retracts, fluid from below the first triangular features 330 and the second triangular feature 332 flows to a portion of the mixing bag 202 above the first triangular features 330 and the second triangular feature 332. The fluid flowing from a lower portion of the mixing bag 202 to an upper portion of the mixing bag 202 flows through a central portion of the mixing bag 202 between the first triangular features 330 and the second triangular feature 332. Because the central region of the mixing bag 202 is constricted by the first triangular features 330 and the second triangular feature 332, the velocity of the fluid is increased compared to if the first triangular features 330 and the second triangular feature 332 were not included in the mixing bag 202. The velocity of the fluid may be high enough to move the fluid from the second end 306 of the mixing bag 202 towards the concave center portion 318 and around the first lobe 310 and the second lobe 312 of the mixing bag 202. In at least one example embodiment, the first triangular feature 330 may have a first triangular edge 334 and the second triangular feature 332 may have a second triangular edge 336. The first triangular edge 334 and the second triangular edge 336 may be the upper edges of the first triangular feature 330 and the second triangular feature 332 and may facilitate fluid movement from the first end 304 to the second end 306 of the mixing bag 202 when fluid is being drained from the mixing bag 202.

The first triangular feature 330 and the second triangular feature 332 may form a first angle 337 of about 34°. However, example embodiments of the first angle 337 are not limited herein. The first triangular feature 330 may couple to the first edge 322 and the second triangular feature 332 may couple to the second edge 324. The first triangular feature 330 and the first edge 322 may form a second angle 338. The second triangular feature 332 and the second edge 324 may also form the second angle 338. In at least one example embodiment, the second angle 338 may be about 110°. However, example embodiments of the second angle 338 are not limited herein.

The mixing bag 202 may include additional rounded corners to facilitate movement of fluids throughout the mixing bag 202. In particular, the mixing bag 202 may include a first rounded edge 340 between the first triangular edge 334 and the first lobe 310 and a second rounded edge 342 between the second triangular edge 336 and the second lobe 312. The first rounded edge 340 and the second rounded edge 342 may have a radius 344 of about 40 mm. However, example embodiments of the radius 344 are not limited herein.

The mixing bag 202 may also include a third rounded edge 350 between the first edge 322 and the first tapered edge 326 and a fourth rounded edge 352 between the second edge 324 and the second tapered edge 328. The third rounded edge 350 and the fourth rounded edge 352 may have a radius 354 of about 20 mm. However, example embodiments of the radius 354 are not limited herein.

The mixing bag 202 may also include a fifth rounded edge 360 between the first lobe 310 and the concave center portion 318 and a sixth rounded edge 362 between the second lobe 312 and the concave center portion 318. The fifth rounded edge 360 and the sixth rounded edge 362 may have a radius 364 of about 20 mm. However, example embodiments of the radius 364 are not limited herein.

In at least one example embodiment, a width 370 of the mixing bag 202 between the first edge 322 and the second edge 324 may be approximately 170 mm. A height 372 of the mixing bag 202 between the fifth rounded edge 360 and the first tapered edge 326 may be approximately 235 mm. The mixing bag 202 may have a port or a mixing bag fluid connection point 374 that may have a height 376 of approximately 7 mm and a width 378 of approximately 6 mm. The mixing bag fluid connection point 374 may be centered along the second end 306 of the mixing bag 202 and may be configured to couple to the mixing bag fluid line 204. While example dimensions of the mixing bag 202 are described herein, the dimensions are not limited to those described herein.

In at least one example embodiment, the mixing bag 202 is configured to hold approximately 500 mL of fluid. The mixing bag 202 may be formed from Ethylene Vinyl Acetate (EVA) in at least one example embodiment.

The example embodiments herein provide improved cell preservation systems and mixing bags. In particular, the cell preservation system 100 may include a first tab 150 and a second tab 152 configured to reduce adhesion between the cooling plate 106 and the mixing bag 202 due to condensation of the cooling plate 106. Also, the mixing bag 202 described herein is configured to unfirmly mix the entire volume of fluid contained therein during a mixing procedure and to drain the fluid from the mixing bag 202 as efficiently as possible to retain as much fluid for storage and transportation as possible. The mixing bag 202 described herein improves the total amount of fluid volume that is able to be processed and facilitates uniform mixing and efficient drainage of the fluid from the mixing bag 202.

While example embodiments and applications of the present invention have been illustrated and described, it is to be understood that the invention is not limited to the precise configuration and resources described above. Various modifications, changes, and variations apparent to those skilled in the art may be made in the arrangement, operation, and details of the methods and systems of the present invention disclosed herein without departing from the scope of the present invention.

It will be apparent to those skilled in the art that various modifications and variations may be made to the methods and structure of the present invention without departing from its scope. Thus, it should be understood that the invention is not to be limited to the specific examples given. Rather, the invention is intended to cover modifications and variations within the scope of the following claims and their equivalents.

What is claimed is:

1. A bag for mixing fluid volumes, the bag comprising:
a port at a center of a first end, the port configured to couple to at least one fluid line for introducing and removing fluids from the bag;
a second end including a first lobe and a second lobe a first constriction on a first side between the first end and the second end; and
a second constriction on a second side between the first end and the second end.

2. The bag of claim 1, wherein the first end is tapered to the port from the first side and the second side.

3. The bag of claim 1, wherein the first lobe and the second lobe are circular.

4. The bag of claim 1, further comprising:
a connection at a center of the second end between the first lobe and the second lobe, the connection being configured to couple with a hanger of a collection system to hold the bag within the collection system.

5. The bag of claim 4, wherein
the first lobe is configured to extend above the connection towards the first side, and
the second lobe is configured to extend above the connection towards the second side.

6. The bag of claim 1, wherein the first constriction includes a first portion and a second portion, each of the first portion and the second portion configured to taper from the second end to the first end of the bag.

7. The bag of claim 1, wherein the second constriction includes a first portion and a second portion, each of the first portion and the second portion configured to taper from the second end to the first end of the bag.

8. A system for mixing fluid volumes, the system comprising:
a bag including
a port at a center of a first end, the port configured to couple to at least one fluid line for introducing and removing fluids from the bag,
a second end including a first lobe and a second lobe,
a first constriction on a first side between the first end and the second end, and
a second constriction on a second side between the first end and the second end
a hanger configured to couple to the second end of the bag to hold the bag in the system; and
at least one mixing paddle configured to interact with the bag to mix the fluid volumes within the bag.

9. The system of claim 8, wherein the first end is tapered to the port from the first side and the second side.

10. The system of claim 8, wherein the first lobe and the second lobe are circular.

11. The system of claim 8, wherein the bag further comprises:
a connector at a center of the second end between the first lobe and the second lobe, the connector being configured to couple with the hanger to hold the bag within the system.

12. The system of claim 11, wherein
the first lobe is configured to extend above the connector towards the first side, and
the second lobe is configured to extend above the connector towards the second side.

13. The system of claim 8, wherein the first constriction includes a first portion and a second portion, each of the first portion and the second portion configured to taper from the second end to the first end of the bag.

14. The system of claim 8, wherein the second constriction includes a first portion and a second portion, each of the first portion and the second portion configured to taper from the second end to the first end of the bag.

15. The system of claim 8, wherein the at least one mixing paddle includes a first mixing paddle and a second mixing paddle.

16. The system of claim 15, wherein the first mixing paddle is configured to pinch the bag to a plate of the system at a central location.

17. The system of claim 16, wherein the central location is between the first constriction and the second constriction such that a first flow path is created between the first constriction and the first mixing paddle and a second flow path is created between the second constriction and the first mixing paddle.

18. The system of claim 15, wherein the second mixing paddle is located between the first mixing paddle and the first end of the bag when the bag is attached to the hanger.

19. The system of claim 15, wherein the second mixing paddle is configured to move from a first location to a second location relative to the bag to mix the fluid volumes within the bag.
